SEQUENCE LISTING (1 sequence)

SEQ ID NO: 1 - DNA, 201 bp, Felis catus
  source 1..201: mol_type = genomic DNA
ttttcctagtgttaagttgccaaaagaaaacagaaacattccctataagttgttcattaaaaacaaaaaacaaaaaaaccaaccgccaacatatggttatdagctcactgctgctggggaaatacaaacccatatgtgttcacagaaaaccgtgagacggaaacaattagggtgattccaaaataatcacctgccgtaaag

(19) United States
(12) Patent Application Publication
(10) Pub. No.: US 2023/0330050 A1
(43) Pub. Date: Oct. 19, 2023

(54) Methods and Compositions for Diagnosis and Treatment of Disorders in Pets

(71) Applicant: Hill's Pet Nutrition, Inc., Topeka, KS (US)

(72) Inventors: Jeffrey BROCKMAN, Lawrence, KS (US); Eden EPHRAIM, Lawrence, KS (US)

(73) Assignee: Hill's Pet Nutrition, Inc., Topeka, KS (US)

(21) Appl. No.: 18/087,426

(22) Filed: Dec. 22, 2022

Related U.S. Application Data
(60) Provisional application No. 63/292,784, filed on Dec. 22, 2021.

(51) Int. Cl. A61K 31/198; A61K 9/00; C12Q 1/6883; A23K 50/40; A23K 20/142

(52) U.S. Cl. CPC A61K 31/198; A61K 9/0056; C12Q 1/6883; A23K 50/40; A23K 20/142; C12Q 2600/156; C12Q 2600/106

(57) ABSTRACT

Aspects of the invention relate to methods for identifying pets prone to disorders and compositions for preventing, mitigating, delaying the onset and/or reducing the severity of conditions or disorders associated with insufficient nitrogen oxide and/or liver disorder. According to one aspect, provided is a method including determining a genotype of the argininosuccinate synthase gene for a subject being a pet, wherein the genotype is determined to be a minor allele, major allele, or heterozygous allele; and providing instructions to administer arginine based on the genotype of the argininosuccinate synthase gene of the subject.

### CROSS-REFERENCE TO RELATED APPLICATION

This application claims the benefit of priority from U.S. Provisional Application No. 63/292,784, entitled “METHODS AND COMPOSITIONS FOR DIAGNOSIS AND TREATMENT OF DISORDERS IN PETS” and filed Dec. 22, 2021, the contents of which is hereby incorporated herein in its entirety.

### SEQUENCE LISTING

The instant application contains a Sequence Listing and has been filed in electronic ST.26 format and is hereby incorporated by reference in its entirety. Said XML file, created on Jul. 10, 2023, is named “Colgate 13107 ST-26 7-10-2023.xml” and is 2,221 bytes in size.

### BACKGROUND

Nitric oxide (“NO”) is a strong vasodilator which helps regulate blood pressure and plays a role in the innate immune system and other immune functions. Nitric oxide is a highly reactive molecule, which complicates its direct measurements. Moreover, nitric oxide is produced at various locations throughout mammalian bodies an involves different enzyme isoforms and non-enzymatic pathways. Further, there are numerous vitamins, minerals, enzymes, amino acids, and other compounds that participate in the production and regulation of nitric oxide. Additional factors that influence bioavailability of nitric oxide include scavenging, or inactivation via hemoglobin, superoxide, or oxidation.

Single nucleotide polymorphisms (“SNPs”) are a common type of genetic variation. SNPs are single base pair mutations at a specific locus. That is, a SNP is a difference in a single nucleotide in a DNA sequence that occurs at a specific position in a genome. Typically, for a SNP at a specific position, there are two possible nucleotide variations, which are referred to as alleles for that position. Within a population, the nucleotide variation that most commonly appears at a specific base position in a genome is referred to as the major allele; the nucleotide variation that is less common at that specific base position is referred to as the minor allele. Pets, like most multicellular organisms have two sets of chromosomes. Thus, each pet has two copies of each gene or locus and therefore two copies of each SNP. Accordingly, for each SNP in a pet's genome, the pet may have two copies of the major allele, one minor allele and one minor allele or two minor alleles.

There is an ongoing need for improved methods and treatments for mammalian animals, such as domestic pets.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

One aspect of the present invention is directed to methods for identifying pets prone to disorders associated with insufficient nitric oxide and/or liver disorders. In certain embodiments, the method comprises determining a genotype of the argininosuccinate synthase gene (ASG) and providing instructions to administer arginine based on the genotype of the ASG. The ASG may be a minor allele, major allele, or heterozygous allele. In some embodiments, the genotype of the argininosuccinate synthase gene is determined from endothelial cells, immune cells, or a combination of two or more thereof.

Another aspect of the present invention is directed to treating or preventing conditions or disorders associated with insufficient nitric oxide with the administration of a diet with high levels of arginine. The diet may consist of a pet food composition or a supplement added to a pet food composition. The diet may be administered through any means known in the art. In some embodiments, the pet food composition is administered orally. In some embodiments, the pet food composition comprises more than 1.44 wt. % of arginine, wherein all weight percentages are based on the total weight of the pet food composition.

In certain embodiments, a method is provided comprising: determining a genotype of the argininosuccinate synthase gene for a subject being a pet, wherein the genotype is determined to be a minor allele, major allele, or heterozygous allele; and providing a composition comprising arginine to the subject when the genotype is determined to be a minor allele or heterozygous allele.

### BRIEF DESCRIPTION OF THE DRAWINGS

The features, and advantages of the invention will be apparent from the following more detailed description of certain embodiments of the invention and as illustrated in the accompanying drawings in which:

FIG. 1 depicts data demonstrating the genotyping of the genetic loci that spans the ASS1 gene in accordance with an aspect of the invention.

FIG. 2 depicts data demonstrating the relative levels of serum citrulline by genotype according to aspects of the invention.

FIG. 3 depicts data demonstrating the prevalence of odontoclastic resorptive lesions in cats based on their genotype in accordance with an aspect of the invention.

FIG. 4 depicts data demonstrating the prevalence of hypertension in cats based on their genotype according to aspects of the invention.

FIG. 5 depicts data demonstrating the comparison between circulating levels of arginine in cats being fed with a high arginine diet and a low arginine diet in accordance with an aspect of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. For example, a range of 0.5-6% would expressly include all intermediate values of, for example, 0.6%, 0.7%, and 0.9%, all the way up to and including 5.95%, 5.97%, and 5.99%, among many others. The same applies to each other numerical property and/or elemental range set forth herein, unless the context clearly dictates otherwise.

Unless otherwise specified, all percentages and amounts expressed herein and elsewhere in the specification should be understood to refer to percentages by weight of the total composition. Unless otherwise specified, reference to an ingredient or component, being present at a “wt. %” and “weight %” refers to the amount of that ingredient or component present in the composition based on the total weight of the composition. When weight percentages are provided based on the total weight of the composition on a dry matter basis, such weight percentages refer to the weight percentage of that ingredient or component in the composition when excluding water.

According to the present application, use of the term “about” in conjunction with a numeral value refers to a value that may be +/−5% of that numeral. As used herein, the term “substantially free” is intended to mean an amount less than about 5.0 weight % (“wt.”), less than 3.0 weight %, 1.0 weight %; preferably less than about 0.5 weight %, and more preferably less than about 0.25 weight % of the composition. As used herein, the term “free” is intended to mean no detectable amount is present.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (i.e. farm cats), pet cats, ferrets, birds, reptiles, rabbits, and fish. The term “feline” is used in reference to “cat” and the terms are used interchangeably.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

The inventors recognized that arginine is an amino acid that participates in the regulation of nitric oxide. In particular, arginine is the substrate for vascular nitric oxide formation and nitric oxide is synthesized from L-arginine by nitric oxide synthase in nearly every cell type. Arginine is normally synthesized from argininosuccinate by argininosuccinase lyase, which is synthesized from citrulline by argininosuccinate synthetase (“ASS1”). The conversion of citrulline to argininosuccinate can be a rate limiting step in arginine synthesis and any delays in this process may directly affect the production of arginine.

The inventors discovered ASS1 is the rate limiting step in Arginine biosynthesis. Moreover, there is a measurable buildup of the ASS1 substrate, citrulline, indicating a decrease in ASS1 activity due to a variant form of the ASS1 gene linked to the minor allele A at SNP D4:90463781 (see Sequence ID No. 1). For instance, it was discovered that felines having the minor allele or the heterozygous allele for the ASS1 gene typically have a buildup of the ASS1 substrate, citrulline, indicating a decrease in ASS1 activity and/or a decreased capacity to produce arginine. Without being limited to any particular theory, it is believed that the SNP for the ASS1 (see Sequence ID No. 1) can serve as a biomarker for the arginosuccinate synthase gene. By way of non-limiting example, the minor allele or heterozygous allele is marked by the A in Sequence ID No. 1.

It is further believed that when the level of arginosuccinate and/or the capacity to produce arginine is decreased, the clinical wellbeing of an animal, such as a pet, is negatively impacted. One aspect of the present invention is directed toward methods for determining pets with a predisposition for developing conditions or disorders related to low nitric oxide synthesis. In some embodiments, the method comprises the step of genotyping a biological sample from a pet, i.e. detecting one or more SNPs in a genetic sample. In some embodiments, the biological sample is obtained from the blood, saliva, follicle root, nasal swab or oral swab of a pet. In some embodiments, SNPs are detected using a method selected from the types of methods consisting of: hybridization-based methods, enzyme-based methods, post-amplification methods based on physical properties of DNA, and sequencing methods. In some embodiments, SNP(s) are detected using methods that include at least one nucleic acid analysis technique selected from: DNA sequencing, restriction enzyme digest, polymerase chain reaction (PCR), hybridization, real-time PCR, reverse transcriptase PCR, or ligase chain reaction.

Another aspect of the present invention is directed toward methods for preventing, delaying the onset and/or reducing the severity of conditions or disorders related to low nitric oxide synthesis stemming from low levels of exogenous arginine. In some embodiments, the methods for preventing, mitigating, delaying the onset and/or reducing the severity of conditions or disorders related to low nitric oxide synthesis stemming from low levels of exogenous arginine comprise identifying the subject as having low arginine synthesis and administering, directly or by adding to the pet's diet, compounds and compositions useful in maintaining exogenous arginine levels. The administration of the compounds and/or compositions for improving or maintaining arginine levels may be via supplementing, adding to, or replacing one or more meals fed to the subject and/or providing a toy configured to release compounds comprising arginine.

In one embodiment, the method further includes administering a food composition for increasing the amount of arginine in a subject. For instance, the method may include providing the subject at least one meal (e.g., two, three, four or more meals) of the food compositions disclosed herein per day. In some cases, the food composition is provided to the subject for at least five days, at least seven days, at least fourteen days, or for at least one month. In at least one embodiment, the subject is provided a food composition disclosed herein at least once a day for one week. Without being limited to a particular theory, the embodiments of the invention advantageously overcomes the inability of a pet to produce sufficient levels of arginine. Additionally, embodiments of the invention may improve the level of arginine in a pet within two days, within one day, within seven hours, or within three hours of consuming certain compositions disclosed herein.

In accordance with another aspect of the invention, provided is a food composition for increasing the level of arginine in a subject. The food compositions disclosed herein may, in some instanced, be administered as part of the methods disclosed herein. The terms “food,” “food composition,” and pet food composition” are used interchangeably and refer to a composition suitable for ingestion by an animal, such as a mammalian animal, and preferably a domestic pet. Pet food compositions may include, without limitation, nutritionally balanced compositions suitable for daily feed, as well as supplements and/or treats, which may or may not be nutritionally balanced. The term “nutritionally balanced” means that a composition, such as a pet food composition, has known required nutrients to sustain life in proper amounts and proportions based on recommendations of recognized authorities, including, but not limited to, governmental agencies and the field of pet nutrition. The pet food composition herein may be a dry composition (for example, kibble), semi-moist composition, wet composition, or any mixture thereof. In certain embodiments, the composition may be a supplement, such as a gravy, drinking water, yogurt, powder, suspension, chew, treat (e.g., biscuits) or any other delivery form.

“Daily nutritional intake” and “total nutritional intake per day” refer to dry matter intake per day. That is, water weight is not included in calculating the amount of nutrition consumed per day. To the extent that food and food ingredient contain water/moisture, the dry matter represents everything in the sample other than water including protein, fiber, fat, minerals, etc. Dry matter weight is the total weight minus the weight of any water. Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day. The skilled artisan would readily recognize and understand nutritional amounts and percentages expressed as dry matter amounts, dry matter weights and dry matter percentages. Since foods, whether wet, moist or dry, generally contain a certain amount of water, when calculating daily dry matter intake, the water component of such food is excluded. To calculate total daily nutritional intake, which is dry matter intake per day, water is excluded. To calculate percent of an ingredient of total daily intake on a dry matter basis, water is removed from the total intake to give total daily dry matter intake and the percent of the ingredient is based on amount of ingredient present as dry matter.

As used herein, an “ingredient” refers to any component of a composition and the term “treatment” refers to eliminating, reducing the severity or preventing one or more symptoms. The term “nutrient” refers to a substance that provides nourishment. In some cases, an ingredient may comprise more than one “nutrient,” for example, a composition may comprise corn comprising important nutrients including both protein and carbohydrate.

The term “effective amount” indicates that the materials or amount of material is effective to achieve the intended result, such as, but not limited to, preventing, alleviating, or ameliorating one or more symptoms of a disease or health condition.

As used herein, the term “kibble” or “food kibble” refers to a particulate pellet like component of cat feeds. In some embodiments, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

As used herein, the term “extrude” or “extrusion” refers to the process of sending preconditioned and/or prepared ingredient mixtures through an extruder. In some embodiments of extrusion, food kibbles are formed by an extrusion processes wherein a kibble dough, including a mixture of wet and dry ingredients, can be extruded under heat and pressure to form the food kibble. Any type of extruder can be used, examples of which include but are not limited to single screw extruders and twin-screw extruders. The list of sources, ingredients, and components as described hereinafter are listed such that combinations and mixtures thereof are also contemplated and within the scope herein.

As used herein, the term “supplement(s)” include, but are not limited to, a feed used with another feed to improve nutritive balance or performance of the total diet for an animal. Supplements include, but are not limited to, compositions that are fed undiluted as a supplement to other feeds, offered free choice with other parts of an animal's ration that are separately available, or diluted and mixed with an animal's regular feed to produce a complete feed. The AAFCO guidelines, for example, contain a discussion relating to supplements in the Official Publication of the Association of American Feed Control Officials, Inc. (AAFCO), Atlanta, Ga. (2012). Supplements may be in various forms including, for example, powders, liquids, syrups, pills, encapsulated compositions and the like.

Sources of proteins, carbohydrates, fats, vitamins, minerals, balancing agents, and the like, suitable for inclusion in the compositions, and particularly in the food products to be administered in methods provided herein, may be selected from among those conventional materials known to those of ordinary skill in the art.

In some embodiments, the kibble may comprise a binder. In certain embodiments the binder includes but is not limited to any of the following or combinations of the following: monosaccharides such as glucose, fructose, mannose, arabinose; di- and trisaccharides such as sucrose, lactose, maltose, trehalose, lactulose; corn and rice syrup solids; dextrins such as corn, wheat, rice and tapioca dextrins; maltodextrins; starches such as rice, wheat, corn, potato, tapioca starches, or these starches modified by chemical modification; alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, high lactose whey by-product, meat broth solids such as chicken broth, chicken broth solids, soy protein, and egg white.

In certain embodiments, the binder includes but is not limited to a lipid and/or lipid derivative. Lipids can be used in combination with water and/or other binder components. Lipids can include plant fats such as soybean oil, corn oil, rapeseed oil, olive oil, safflower oil, palm oil, coconut oil, palm kernel oil, and partially and fully hydrogenated derivatives thereof; animal fats and partially and fully hydrogenated derivatives thereof; and waxes.

The composition of the present disclosure can additionally comprise other additives in amounts and combinations familiar to one of skill in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may include, by way of example, substances that tend to increase the shelf life of the composition. Other examples of other such additives potentially suitable for inclusion in the compositions of the invention include, for example, preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Additives for coloring, palatability, and nutritional purposes can include colorants, salts (including, but not limited to, sodium chloride, potassium citrate, potassium chloride, and other edible salts), vitamins, minerals, and flavoring. Other additives can include omega-3 fatty acids, omega-6 fatty acids, glucosamine, chondroitin sulfate, vegetable extracts, herbal extracts, etc.

The concentration of such additives in the composition typically can be up to about 5% by weight, based on the total weight of the composition on a dry matter basis. In some embodiments, the concentration of such additives (particularly where such additives are primarily nutritional balancing agents, such as vitamins and minerals) is from about 0% to about 2.0% by weight, based on the total weight of the composition on a dry matter basis. In some embodiments, the concentration of such additives (again, particularly where such additives are primarily nutritional balancing agents) is from about 0% to about 1.0% by weight, based on the total weight of the composition on a dry matter basis. Although the list of foregoing additives may be potentially suitable in some embodiments, one or more of the foregoing additives may be excluded from other embodiments of the composition.

The pet food composition may comprise protein and/or a digestible crude protein. The term “protein” means a polypeptide, or a peptide, or a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompasses polymers of any length, including polymers containing from about 2 to 1000, from 4 to 800, from 6 to 600, and from 8 to 400 amino acids. The term includes amino acid polymers that are synthesized and that are isolated and purified from natural sources. Under some embodiments, the terms “polypeptide”, “peptide” or “protein” are used interchangeably.

“Digestible crude protein” is the portion of protein that is available or can be converted into free nitrogen (amino acids) after digesting with gastric enzymes. In vitro measurement of digestible crude protein may be accomplished by using gastric enzymes, such as pepsin, to digest a sample and measure the free amino acid after digestion. In vivo measurement of digestible crude protein may be accomplished by measuring the protein levels in a feed/food sample and feeding the sample to an animal and measuring the amount of nitrogen collected in the animal's feces.

The protein and/or digestible crude protein of the composition may be present at various amounts or concentrations. In one embodiment, protein may be present in an amount of from about 10% to about 40%, based on the total weight of the pet food composition. For example, protein may be present in an amount of about 10 weight %, about 15 weight %, about 20 weight %, about 25 weight %, about 30 weight %, about 35 weight %, or about 40 weight %. In another example, protein may be present in an amount of from about 10% to about 25%, from about 15% to about 25%, or about 15% to about 20%, based on the total weight of the pet food composition on a dry matter basis. In certain embodiments, protein is present in an amount of about 12% to about 35%, about 13% to about 25%, or about 15% to about 25%, based on the total weight of the pet food composition on a dry matter basis.

A portion of the protein in the composition may be digestible protein. For example, the composition may include an amount of protein, where about 40 weight % or more, about 50 weight % or more, about 60 weight % or more, about 70 weight % or more, about 80 weight % or more, about 90 weight % or more, about 95 weight % or more, about 98 weight % or more, or about 99 weight % or more of the protein is digestible protein. In some embodiments, e.g., when the composition desirable promotes weight loss, the portion of protein that is digestible protein is about 60 weight % or less, about 50 weight % or less, about 40 weight % or less, about 30 weight % or less, about 20 weight % or less, or about 10 weight % or less, based on the total amount of protein in the composition. In further embodiment, the amount of protein that is digestible protein is about 10 to about 99 weight %, about 10 to about 95 weight %, about 10 to about 90 weight %, about 10 to about 70 weight %, about 10 to about 50 weight %, about 10 to about 30 weight %; about 30 to about 99 weight %, about 30 to about 95 weight %, about 30 to about 90 weight %, about 30 to about 70 weight %, about 30 to about 50 weight %; about 50 to about 99 weight %, about 50 to about 95 weight %, about 50 to about 90 weight %, about 50 to about 70 weight %; or about 70 to about 99 weight %, about 70 to about 95 weight %, about 70 to about 90 weight %, including ranges and subranges therein, based on the total amount of protein in the composition.

Protein may be supplied by any of a variety of sources known by those of ordinary skill in the art including plant sources, animal sources, microbial sources or a combination of these. For example, animal sources may include meat, meat-by products, seafood, dairy, eggs, etc. Meats, for example, may include animal flesh such as poultry, fish, and mammals including cattle, pigs, sheep, goats, and the like. Meat by-products may include, for example, lungs, kidneys, brain, livers, stomachs and intestines. Plant protein includes, for example, soybean, cottonseed, and peanuts. Microbial sources may be used to synthesize amino acids (e.g., lysine, threonine, tryptophan, methionine) or intact protein such as protein from sources listed below.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

In some embodiments, the food composition further comprises one or more amino acids. Amino acids, including essential amino acids, can be added to the compositions of the present disclosure as free amino acids, or supplied by any number of sources, e.g., crude protein, to the compositions of the present disclosure. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan, histidine and arginine. In addition, taurine, while technically not an amino acid but a derivative of cysteine, is a nutrient for cats and, thus, may be included in certain food compositions disclosed herein.

Examples of amino acids that may be included in the pet food composition include, but are not limited to, Tryptophan, Taurine, Histidine, Carnitine, Carnosine, Alanine, Cysteine, Arginine, Methionine (including DL-methionine, and L-methionine), Tryptophan, Lysine, Asparagine, Aspartate (Aspartic acid), Phenylalanine, Valine, Threonine, Isoleucine, Histidine, Leucine, Glycine, Glutamine, Taurine, Tyrosine, Homocysteine, Ornithine, Citruline, Glutamate (Glutamic acid), Proline, and/or Serine. Sources of carotenoids may include lutein, astaxanthin, zeaxanthin, bixin, lycopene, and/or beta-carotene. Sources of antioxidant ingredients may comprise tocopherols (vitamin E), vitamin C, vitamin A, plant-derived materials, carotenoids (described above), selenium, and/or CoQ10 (Co-enzyme Q10). In a preferred embodiment, the pet food composition contains high levels of arginine and derivatives thereof. The amount of arginine present in the composition may be about 0.01 to about 10.0 weight %, about 0.01 to about 5.0 weight %, about 0.01 to about 2.0 weight %, about 0.1 to about 10.0 weight %, about 0.1 to about 5.0 weight %, about 0.1 to about 2.0 weight %; about 0.5 to about 5.0 weight %, about 0.5 to about 2.0 weight %, about 1.5 to about 5.0 weight %, about 1.5 to about 2.0 weight %, about 0.5 weight %, about 1.0 weight %, about 1.4 weight%, about 1.44 weight %, about 1.8 weight %, or about 2.0 weight %, based on the total weight of the composition on a dry matter basis. The arginine present in the composition may L-arginine, D-arginine, or a mixture thereof.

The term “carbohydrate” as used herein includes polysaccharides (e.g., starches and dextrins) and sugars (e.g., sucrose, lactose, maltose, glucose, and fructose) that are metabolized for energy when hydrolyzed. One skilled in the art could manipulate the texture of the final product by properly balancing carbohydrate sources. For example, short chain polysaccharides lend to be sticky and gluey, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional additives is at least partially dependent on the nutritional requirements for different life stages of animals.

Carbohydrates can be supplied by any of a variety of sources known by those skilled in the art, including, but not limited to, oat fiber, cellulose, peanut hulls, beet pulp, parboiled rice, corn starch, corn gluten meal, cereal, and sorghum. Grains supplying carbohydrates can include, but are not limited to, wheat, durum, semolina, corn, barley, and rice. In certain embodiments, the carbohydrate component comprises a mixture of one or more carbohydrate sources. Carbohydrates content of foods can be determined by any number of methods known by those of skill in the art.

Generally, the carbohydrate percentage can be calculated as nitrogen free extract (“NFE”), which can be calculated as follows: NFE=100%-(moisture %)-(protein %)-(fat %)-(ash %)-(crude fiber %). The amount of carbohydrate, e.g., calculated as NFE, present in the composition may be about 10 to about 90 weight %, about 10 to about 70 weight %, about 10 to about 50 weight %, about 10 to about 40 weight %, about 10 to about 30 weight %, about 10 to about 20 wt. %; about 20 to about 90 weight %, about 20 to about 70 weight %, about 20 to about 50 weight %, about 20 to about 40 weight %; about 30 to about 90 weight %, about 30 to about 70 weight %, about 30 to about 50 weight %, about 30 to about 40 weight %; about 50 to about 90 weight %, about 50 to about 70 weight %; or about 70 to about 90 weight %, based on the total weight of the composition on a dry matter basis.

Dietary fiber refers to components of a plant which are resistant to digestion by an animal's digestive enzymes. Dietary fiber includes soluble and insoluble fibers. Soluble fibers are resistant to digestion and absorption in the small intestine and undergo complete or partial fermentation in the large intestine and may be chosen from, e.g., beet pulp, guar gum, chicory root, psyllium, pectin, blueberry, cranberry, squash, apples, oats, beans, citrus, barley, or peas. Insoluble fibers can be supplied by any of a variety of sources, including, for example, cellulose, whole wheat products, wheat oat, corn bran, flax seed, grapes, celery, green beans, cauliflower, potato skins, fruit skins, vegetable skins, peanut hulls, and soy fiber. Crude fiber includes indigestible components contained in cell walls and cell contents of plants such as grains, for example, hulls of grains such as rice, corn, and beans. Typical crude fiber amounts in compositions of the present disclosure can be from about 0 to 10 weight %, about 1 to about 5 weight %, about 1 to about 3 weight %, or about 1 to about 2 weight %, based on the total weight of the pet food composition on a dry matter basis.

The pet food composition may comprise a total dietary fiber. The total dietary fiber may be present at various amounts or concentrations. In one embodiment, total dietary fiber may be present in an amount of less than 20%, based on the total weight of the pet food composition. In certain embodiments, the total dietary fiber is present in an amount of about 10% to about 20%, based on the total weight of the pet food composition. For example, total dietary fiber may be present in an amount of about 10.0 weight %, about 10.5 weight %, about 11.0 weight %, about 11.5 weight %, about 12.0 weight %, about 12.5 weight %, about 13.0 weight %, about 13.5 weight %, about 14.0 weight %, about 14.5 weight %, about 15.0 weight %, about 15.5 weight %, about 16.0 weight %, about 16.5 weight %, about 17.0 weight %, about 17.5 weight %, about 18.0 weight %, about 18.5 weight %, about 19.0 weight %, about 19.5 weight %, or about 20.0 weight %. In another example, total dietary fiber may be present in an amount of from about 10% to about 18%, about 12% to about 18%, or about 15% to about 18%, based on the total weight of the pet food composition. In further embodiments, total dietary fiber is present in an amount of about 15% to about 20%, about 16% to about 19%, or about 16% to about 18%, based on the total weight of the pet food composition on a dry matter basis.

The compositions of the present invention may optionally comprise fat. The term “fat” generally refers to a lipid or mixture of lipids that may generally be a solid or a liquid at ordinary room temperatures (e.g., 25° C.) and pressures (e.g., 1 atm). In some instances, the fat may be a viscous liquid or an amorphous solid at standard room temperature and pressure. The fat may be incorporated completely within the food composition, deposited on the outside of the food composition, or a mixture of the two methods. In some embodiments, the compositions further include an effective amount of one or more substances selected from the group consisting of glucosamine, chondroitin, chondroitin sulfate, methylsulfonylmethane (“MSM”), creatine, antioxidants, Perna canaliculata, omega-3 fatty acids, omega-6 fatty acids and mixtures thereof.

Fat can be supplied by any of a variety of sources known by those skilled in the art, including meat, meat by-products, canola oil, fish oil such as anchovy oil and menhaden oil, and plants. Meat fat sources include poultry fat, turkey fat, pork fat, lard, tallow, and beef fat. Plant fat sources include wheat, flaxseed, rye, barley, rice, sorghum, corn, oats, millet, wheat germ, corn germ, soybeans, peanuts, and cottonseed, as well as oils derived from these and other plant fat sources such as corn oil, soybean oil, cottonseed oil, palm oil, palm kernel oil, linseed oil, canola oil, rapeseed oil, and/or olestra.

The compositions of the present disclosure may contain at least about 9 weight % (or from about 9 to about 25 weight %, or from about 10 to about 20 weight %, or from about 10 to about 15 weight %) of total fat, based on the total weight of the pet food composition. In some cases, the fat in the compositions is crude fat. Crude fat may be included into the compositions in amounts of from about 10 to about 20 weight %, about 10 to about 18 weight %, about 10 to about 16 weight %; about 12 to about 20 weight %, about 12 to about 18 weight %, or about 12 to about 16 weight %, based on the total weight of the composition on a dry matter basis. In some cases, it may be preferable that about 50 weight % or more, about 60 weight % or more, about 70 weight % or more, about 80 weight % or more, or about 90 weight % or more of the total fat is obtained from an animal source. Alternatively, about 50 weight % or more, about 60 weight % or more, about 70 weight % or more, about 80 weight % or more, or about 90 weight % or more of the total fat may be obtained from a plant source.

In some embodiments, the food composition further comprises one or more fatty acids such as but not limited to lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, arachidonic acid, alpha-linolenic acid, gamma linolenic acid, linoleic acid, eicosapentanoic acid (EPA), docosahexanoic acid (DHA), and/or fish oils as a source of EPA and/or DHA. Sources of glucose mimetics may comprise glucose anti-metabolites including 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose.

In some embodiments, the food composition further comprises one or more micronutrients including, but not limited to, beta-carotene, alpha-lipoic acid, glucosamine, chondroitin sulfate, lycopene, lutein, and quercetin.

The probiotic component may comprise any suitable bacteria, yeast, microorganisms, and/or mixtures of any thereof. Various probiotic microorganisms are known in the art. In certain embodiments, the probiotic component may comprise bacteria of the order Lactobacillales; bacteria of the genus Bacillus, Bacteroides, and/or Bifidobacterium; yeast of the order Saccharomycetales including the genus Saccharomyces and Candida; and/or mixtures of any thereof. The probiotic may or may not form a spore.

In certain embodiments, the pet food composition comprises vitamins, minerals, and trace elements in amounts required to avoid deficiency and maintain health. These amounts are readily available in the art. Minerals may specifically be maintained at optimum levels known by those skilled in the art to reduce the incidence of stone formation. For example, the Official Publication of the Associate of American Feed Control Officials, Inc. (“AAFCO”), Nutrient Requirements of Dogs and Cats, 2006 provides recommended amounts of such ingredients for dogs and cats.

Minerals and trace elements may include, but are not limited to, boron, calcium, chlorine, chromium, cobalt, copper, fluorine, iodine, iron, magnesium, manganese, molybdenum, phosphorous, potassium, selenium, sodium, sulfur, and zinc. Mineral sources can include, for example, sodium selenite, monosodium phosphate, calcium carbonate, potassium chloride, ferrous sulfate, zinc oxide, manganese sulfate, copper sulfate, manganous oxide, potassium iodide, and/or cobalt carbonate. Typical mineral amounts are about 0.1% to about 4% or about 1% to about 2%.

Vitamins may include, but are not limited to, vitamin A, vitamin B₁ (thiamine or related sources such as thiamine mononitrate), vitamin B₂ (riboflavin), vitamin B₃ (niacin), vitamin B₅ (pantothenic acid or related sources such as calcium pantothenate), vitamin B₆ (pyridoxine or related sources such as pyridoxine hydrochloride), vitamin B₈ (folic acid), vitamin B₁₂, vitamin C (ascorbic acid), vitamin D (such as a vitamin D3 supplements), vitamin E, vitamin H (biotin), vitamin K, acetate, choline and choline related sources such as choline chloride, and inositol. Typical vitamin amounts in the composition of the invention are about from 0% to about 3% or about 1% to about 2%, based on the total weight of the composition on a dry matter basis.

In certain embodiments, the pet food composition may include polyphenols. In some embodiments, the polyphenol source comprises a phenolic compound selected from ellagic acid; gallic acid; protocatechuic acid; p-hydroxybenzoic acid; catechin; and a combination of two or more thereof. In some embodiments, the polyphenol source comprises pecan shells, or any other component of the pecan nut. Examples of further sources of polyphenols may comprise tea extract, rosemary extract, rosemarinic acid, coffee extract, pecan shells, caffeic acid, turmeric extract, blueberry extract, grape extract, grapeseed extract, and/or soy extract

Still other ingredients may include beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Lcarnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, and/or keratolytics.

The pet food compositions disclosed herein may be wet or dry compositions, and the propolis and other ingredients can be either incorporated into the food composition or on the surface of any composition component, such as, for example, by spraying, agglomerating, dusting, or precipitating on the surface.

In certain aspects, pet food compositions of any consistency or moisture content are contemplated, e.g., the compositions of the present invention can be, for example, a dry, moist or semi-moist animal food composition. “Semi-moist” refers to a food composition containing from about 25 to about 35% moisture. “Moist” food refers to a food composition that has a moisture content of about 60 to 90% or greater. “Dry” food refers to a food composition with about 3 to about 12% moisture content and is often manufactured in the form of small bits or kibbles. The food products may also include components of more than one consistency, for example, soft, chewy meat-like particles or pieces as well as kibble having an outer cereal component or coating and an inner “cream” component.

In the pet food industry, foods are generally classified as “wet” or “dry.” A wet food has a relatively high amount of water and is usually present in a can or a container wherein air is substantially or totally excluded. Examples of such foods are “chunk and gravy,” individual solid particles in the presence of liquid gravy or a loaf type material which generally takes the shape of the receptacle. A dry food is generally a baked or extruded material, the latter then cut into individual shaped portions, usually known as kibbles. Propolis can be readily incorporated into a wet or dry food through conventional means.

In certain embodiments, the pet food composition comprises moisture. The moisture may be present at various amounts or concentrations. In one embodiment, moisture may be present in an amount of from about 5% to about 15%, based on the total weight of the pet food composition. For example, moisture may be present in an amount of about 5.0 weight %, about 5.5 weight %, about 6.0 weight %, about 6.5 weight %, about 7.0 weight %, about 7.5 weight %, about 8.0 weight %, about 8.5 weight %, about 9.0 weight %, about 9.5 weight %, about 10.0 weight %, about 10.5 weight %, about 11.0 weight %, about 11.5 weight %, about 12.0 weight %, about 12.5 weight %, about 13.0 weight %, about 13.5 weight %, about 14.0 weight %, about 14.5 weight %, or about 15.0 weight %. In another example, moisture may be present in an amount of from about 6% to about 12%, about 9% to about 13%, about 9% to about 11%, or about 9% to about 13%, based on the total weight of the pet food composition. In certain embodiments, moisture is present in an amount of about 5% to about 12%, about 6% to about 11%, or about 7% to about 10.0%, based on the total weight of the pet food composition. In further embodiments, moisture is present in an amount of about 65% to about 85%, about 60% to about 80%, or about 60% to about 75%, based on the total weight of the pet food composition.

In certain embodiments, the pet food is in a dry form. In certain embodiments, the pet food is a kibble. In certain embodiments, the kibble is dry. In other embodiments, the kibble comprises moisture.

In some embodiments, the composition may be prepared in a canned or wet form using conventional food preparation processes known to those of ordinary skill in the art. Typically, ground animal proteinaceous tissues are mixed with the other ingredients, such as cereal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture is heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The compositions and food products of the present invention can also be added to or combined with food compositions before, during, or after their preparation.

In some embodiments, the food products may be prepared in a dry form using convention processes known to those of ordinary skill in the art. Typically, dry ingredients, including dried animal protein, plant protein, grains and the like are ground and mixed together. Liquid or moist ingredients, including fats, oils water, animal protein, water, and the like are added combined with the dry materials. The specific formulation, order of addition, combination, and methods and equipment used to combine the various ingredients can be selected from those known in the art. For example, in certain embodiments, the resulting mixture is process into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a composition, any ingredient generally may be incorporated into the composition during the processing of the formulation, e.g., during and/or after mixing of the other components of the composition. Distribution of these components into the composition can be accomplished by conventional means. In certain embodiments, ground animal and/or poultry proteinaceous tissues are mixed with other ingredients, including nutritional balancing agents, inorganic salts, and may further include cellulose, beet pulp, bulking agents and the like, along with sufficient water for processing.

In some embodiments, the compositions are formulated so as to be easier to chew. In specific embodiments, the compositions and food products are formulated to address specific nutritional differences between species and breeds of animals, as well as one of more of the attributes of the animal. For example, cat foods, for example, are typically formulated based upon the life stage, age, size, weight, body composition, and breed.

In another embodiment, treats comprising effective amounts of arginine can be prepared by, for example, an extrusion or baking process. Treats include, for example, compositions that are given to an animal to entice the animal to eat during a non-meal time. Treats may be nutritional, wherein the composition comprises one or more nutrients, and may, for example, have a composition as described above for food. Non-nutritional treats encompass any other treats that are non-toxic. Compositions can be coated onto the treat, incorporated into the treat, or both.

In another embodiment, an animal toy is provided that is a chewable or consumable toy. Such toys are typically prepared by coating any existing toy with effective amounts of arginine. Toys therefore include, for example, chewable toys. In certain embodiments, the composition of the invention can form a coating on the surface of the toy or on the surface of a component of the toy, or it can be incorporated partially or fully throughout the toy, or both. It should be recognized that this invention contemplates both partially consumable toys and fully consumable toys.

The pet food composition may be determined by any of the variety of methods for feed analysis known by one skilled in the art. Feed analysis may be done to measure any of the nutritional content listed herein including moisture, protein, fiber, carbohydrate, energy, vitamin, mineral, energy, fat, and ash content.

Protein content may be measured and reported in any of the variety of methods known to one skilled in the art. Protein may be reported as crude protein (CP) to measure both true protein content and non-protein nitrogen. Crude protein content may be further differentiated between degradable intake protein (DIP), undegradable intake protein (UIP) and metabolizeable protein (MP). In certain embodiments, protein content may be differentiated to include heat damaged protein or insoluble crude protein (ICP), adjusted crude protein (ACP), and digestible protein (DP).

Fiber content may be measured and reported in any of the variety of methods known to one skilled in the art. Fiber content may be reported as total dietary fiber (TDF, a combination of soluble and insoluble fiber) crude fiber (CF), neutral detergent fiber (NDF), acid detergent fiber (ADF) and/or acid detergent lignin (ADL). Crude fiber is generally known to estimate the indigestible portion of plant material found in pet food compositions. ADF measures cellulose and lignin, components of plant cell walls. NDF measures the total material found in plant cell walls and includes hemicellulose in addition to the fiber content measured as ADF. ADL measures only the lignin portion of a plant cell wall.

Energy content may be measured and reported in any of the variety of methods known to one skilled in the art. Energy content may be reported as digestible energy (DE), metabolizable energy (ME), net energy (NE), total digestible nutrient (TDN), ether extract (EE), relative feed value (RFV), and relative forage quality (RFQ).

In accordance with a first embodiment, provided is a method for identify pets prone to disorders associated with insufficient nitrogen oxide and/or liver disorder, the method comprising: determining a genotype of the argininosuccinate synthase gene for a subject, the subject being a pet, wherein the genotype is determined to be a minor allele, major allele, or heterozygous allele; and providing instructions to administer arginine based on the genotype of the argininosuccinate synthase gene of the subject.

According to a second embodiment, the method of the first embodiment, wherein the genotype of the argininosuccinate synthase gene is determined from endothelial cells, immune cells, or a combination of two or more thereof.

According to a third embodiment, the method of the any foregoing embodiment further comprising providing a pet food composition comprising more than 1.44 wt. % of arginine, wherein all weight percentages are based on the total weight of the pet food composition.

According to a fourth embodiment, the method of the any foregoing embodiment, wherein the subject is a domestic pet.

According to a fifth embodiment, the method of the any foregoing embodiment, wherein the subject is a feline.

According to a sixth embodiment, the method of the any foregoing embodiment, wherein the genotype is determined using hybridization-based methods, enzyme-based methods, post-amplification methods based on physical properties of DNA, and sequencing methods.

According to a seventh embodiment, the method of the any foregoing embodiment, wherein the genotype is determined to be a minor allele, major allele, or heterozygous allele using at least one nucleic acid analysis technique selected from: DNA sequencing, restriction enzyme digest, polymerase chain reaction (PCR), hybridization, real-time PCR, reverse transcriptase PCR, ligase chain reaction, and a combination of two or more thereof.

In accordance with an eighth embodiment, a method is provided for identifying pets prone to disorders associated with insufficient nitrogen oxide and/or liver disorder, the method comprising: determining a genotype of the argininosuccinate synthase gene for a subject being a pet, wherein the genotype is determined to be a minor allele, major allele, or heterozygous allele; and providing a composition comprising arginine to the subject when the genotype is determined to be a minor allele or heterozygous allele.

According to a ninth embodiment, the method of embodiment eight, wherein the composition is administered orally.

According to a tenth embodiment, the method of embodiment eight or nine, wherein the composition is a food composition.

According to an eleventh embodiment, the method of embodiment ten, wherein the food composition comprises more than 1.44 wt. % of arginine, wherein all weight percentages are based on the total weight of the pet food composition.

According to a twelfth embodiment, the method of any of embodiment eight to eleven, wherein the genotype of the argininosuccinate synthase gene is determined from endothelial cells, immune cells, or a combination of two or more thereof.

According to a thirteenth embodiment, the method of any of embodiment eight to twelve, wherein the genotype is determined using a hybridization-based method, an enzyme-based method, a post-amplification method based on physical properties of DNA, a sequencing method, or a combination of two or more thereof.

According to a fourteenth embodiment, the method of any of embodiment eight to thirteen, wherein the genotype is determined to be a minor allele, major allele, or heterozygous allele using at least one nucleic acid analysis technique selected from: DNA sequencing, restriction enzyme digest, polymerase chain reaction (PCR), hybridization, real-time PCR, reverse transcriptase PCR, ligase chain reaction, and a combination of two or more thereof.

According to a fifteenth embodiment, the method of any of embodiment eight to fourteen further comprising: providing instructions to provide a composition comprising arginine based on the genotype of the argininosuccinate synthase gene of the subject.

### EXAMPLES

### Example 1

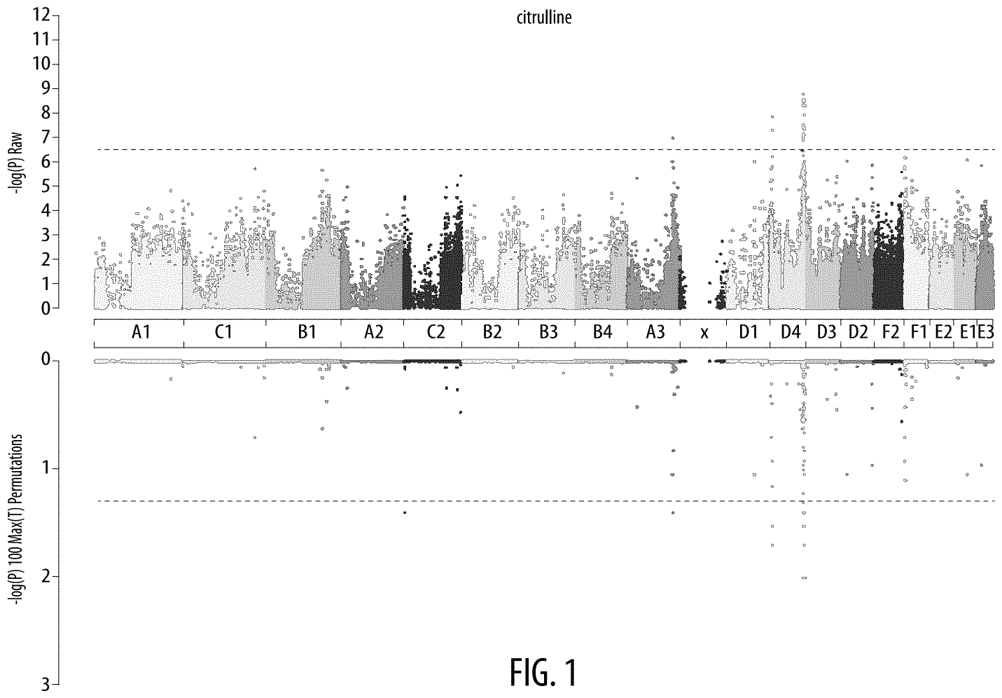

A study of 429 randomly bred cats was conducted to evaluate the use of serum citrulline levels for determining the genotype of the argininosuccinate synthase gene. It is believed that the most significant single nucleotide polymorphism (“SNP”) lies approximately 200 Kb upstream of the argininosuccinate synthase gene start site for transcription, at base position 90463781 on chromosome D4. The major allele has a reference base G and the minor allele has a reference base A, with MAF of 0.31 (see FIG. 1). The DNA sequence of the SNP and flanking sequence, which were assessed using FelCat9.0 reference genome (negative strand) are shown in SEQ ID NO: 1. Semi-quantitative levels of serum citrulline were determined using metabolite profiling by Metabolon, Inc.

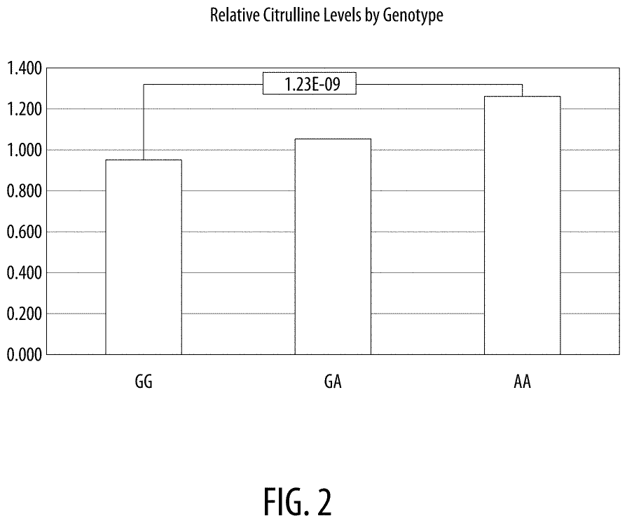

It was determined that the average normalized citrulline values for cats having the homozygous minor allele (“AA”) or the homozygous major allele (“GG”) was 1.27 and 0.96, respectively. The heterozygous cats (AG) had an average normalized citrulline level of 1.08. While the average difference between cats having homozygous minor and major alleles was only 31%, the difference was highly significant as the p-value was determined to be 1.23e-09 (see FIG. 2, Table 1). Because the AA genotype exhibits a buildup of the argininosuccinate synthase substrate citrulline, it is believed that this variant tags a causative mutation that either results in an altered protein structure or impacts a regulatory element that results in decreased expression of argininosuccinate synthase. Without being limited to any specific theory, these homozygous subjects have a lower capacity to produce arginine through the arginine synthesis pathway.

TABLE 1

| | G11 | G12 | G22 |
|---|---|---|---|
| Genotype | A/A | A/G | G/G |
| Counts | 50 | 211 | 168 |
| Frequency | 0.1166 | 0.4918 | 0.3916 |
| Mean | 1.272 | 1.079 | 0.9615 |
| SD | 0.374 | 0.3187 | 0.266 |

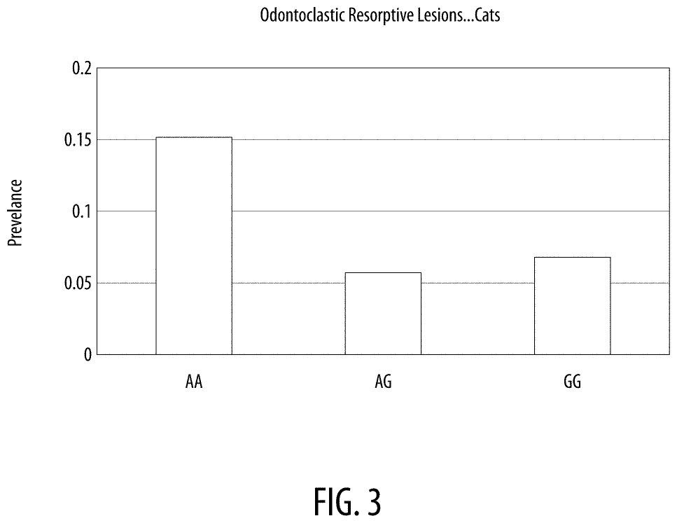

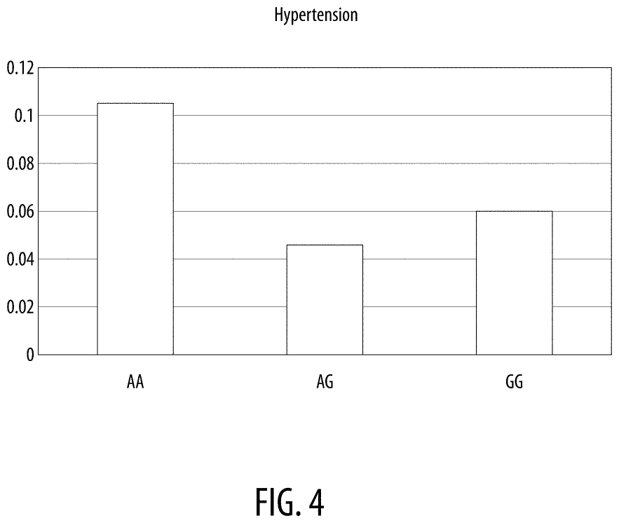

To determine if the AA genotype had any clinical effects, the 429 cats were screened for higher incidences of clinical outcomes in AA cats compared to cats with the GG or AG genotypes. It was determined that cats with the AA genotype have a 250% increase in the incidence rate of odontoclastic resorptive lesions and an about 200% increase in the rate of incidence for hypertension relative to cats with either the AG or GG genotypes (see FIGS. 3 and 4). Even though the serum levels of citrulline in the AG genotype cats were higher than in the GG genotype cats, the difference in incidence rates of odontoclastic resorptive lesions and hypertension was not statistically significant.

The foregoing findings support the conclusion that argininosuccinate synthase has an impact on nitric oxide levels in tissues. For instance, limiting nitric oxide in endothelia tissues under certain conditions is a risk factor for the clinical manifestation of hypertension because nitric oxide is vasodilator. Additionally, nitric oxide is a key signaling molecule to tell the odontoclast to release from the bone. Odontoclast are specialized macrophages that remove tooth bone material in contrast to odontoblast that build tooth bone material. Thus, if nitric oxide is limited, it can result in over activity of the odontoclast resulting in a higher risk of developing resorptive lesions. Accordingly, it is believed that argininosuccinate synthase can impact nitric oxide levels in tissues.

### Example 2

A study was conducted on 40 cats to evaluate the benefit of feeding an exemplary pet food composition (Example Composition A) having a higher level of arginine as compared to comparative pet food composition (Comparative Composition 1). Example Composition A contained 1.84 wt. % of arginine, while Comparative Composition 1 had 1.44 wt. % of arginine, where the weight percentages were based on the total weight of the respective pet food compositions. The formulations for Example Composition A and Comparative Composition 1 are provided in Table 2 (below).

TABLE 2

| | Example | Comparative |
|---|---|---|
| | Composition A | Composition 1 |
| Ingredients | (wt. %) | (wt. %) |
| Rice, brown | 24.7 | — |
| Corn, gluten, meal | 14.5 | 22.5 |
| Pea, protein concentrate | 14.5 | — |
| Oat, groats | 8 | — |
| Chicken Fat acidified | 7.1 | — |
| Chicken Dried 10% Ash | 7 | 9.8 |
| Palatant | 2.5 | — |
| Lactic acid, blend 84% | 1.2 | 1.2 |
| Soybean oil, crude, degummed | 1 | 0.5 |
| Calcium sulfate | 1 | 0.8 |
| Methionine, dl | 0.7 | — |
| Choline chloride, liquid, 70% | 0.6 | 0.4 |
| Sodium chloride, iodized | 0.5 | <0.1 |
| Fish oil, TG, 18/12, NP | 0.5 | 1 |
| Carnitine, 1, 10% | 0.5 | 0.5 |
| Potassium chloride | 0.4 | 0.6 |
| Taurine | 0.3 | 0.2 |
| Vitamin E, oil, 29% | 0.3 | 0.3 |
| Vitamin Premix | 0.2 | 0.1 |
| L-Cysteine hydrochloride | 0.1 | — |
| monohydrate | | |
| Mineral, premix, 2305 | <0.1 | 0.1 |
| Chicken, grd, fresh, Nat | 12.5 | — |
| Feline Anti-Aging Blend | 1.1 | — |
| Beet, pulp, pelleted | 0.9 | — |
| Wheat, red, whole | | 22.6 |
| Rice, brewers | | 15.3 |
| Pork Fat | | 11.7 |
| Egg Dried pelleted | | 5 |
| Chicken Liver Digest | | 2.5 |
| Flax seed whole brown | | 2.3 |
| Wheat, gluten | | 2.2 |
| Calcium carbonate | | 0.3 |
| Total | 100 | 100 |

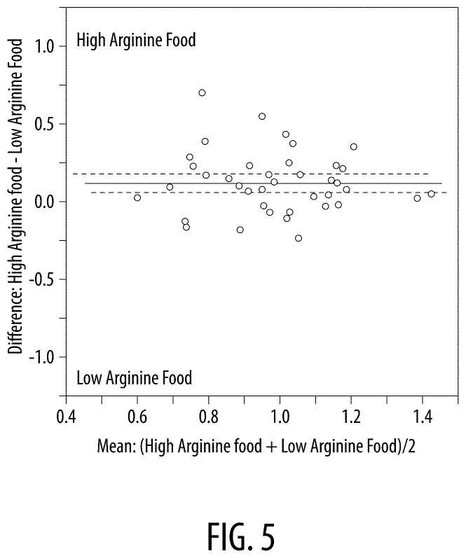

The relative levels of arginine were measured by plasma metabolomics after the cats were fed either Example Composition A or Comparative Composition 1 for 30 consecutive days. The cats that received Example Composition A exhibited a significant increase in circulating levels of arginine (P=0.0008; SE=0.0004) (see FIG. 5). FIG. 5 shows that, assuming endogenous arginine levels are suboptimal for good health in cats with the A genotype, the levels of arginine in the feline body can be increased through an exogenous source such as a food, supplement, etc.

What is claimed is:

1. A method for identifying pets prone to disorders associated with insufficient nitrogen oxide and/or liver disorder, the method comprising:
determining a genotype of the argininosuccinate synthase gene for a subject, the subject being a pet, wherein the genotype is determined to be a minor allele, major allele, or heterozygous allele; and
providing instructions to administer arginine based on the genotype of the argininosuccinate synthase gene of the subject.

2. The method of claim 1, wherein the genotype of the argininosuccinate synthase gene is determined from endothelial cells, immune cells, or a combination of two or more thereof.

3. The method of claim 1 further comprising:
providing a pet food composition comprising more than 1.44 wt. % of arginine, wherein all weight percentages are based on the total weight of the pet food composition.

4. The method of claim 1, wherein the subject is a domestic pet.

5. The method of claim 1, wherein the subject is a feline.

6. The method of claim 1, wherein the genotype is determined using hybridization-based methods, enzyme-based methods, post-amplification methods based on physical properties of DNA, and sequencing methods.

7. The method of claim 1, wherein the genotype is determined to be a minor allele, major allele, or heterozygous allele using at least one nucleic acid analysis technique selected from: DNA sequencing, restriction enzyme digest, polymerase chain reaction (PCR), hybridization, real-time PCR, reverse transcriptase PCR, ligase chain reaction, and a combination of two or more thereof.

8. A method for identifying pets prone to disorders associated with insufficient nitrogen oxide and/or liver disorder, the method comprising:
determining a genotype of the argininosuccinate synthase gene for a subject being a pet, wherein the genotype is determined to be a minor allele, major allele, or heterozygous allele; and
providing a composition comprising arginine to the subject when the genotype is determined to be a minor allele or heterozygous allele.

9. The method of claim 8, wherein the composition is administered orally.

10. The method of claim of claim 8, wherein the composition is a food composition.

11. The method of claim 10, wherein the food composition comprises more than 1.44 wt. % of arginine, wherein all weight percentages are based on the total weight of the pet food composition.

12. The method of claim 8, wherein the genotype of the argininosuccinate synthase gene is determined from endothelial cells, immune cells, or a combination of two or more thereof.

13. The method of claim 8, wherein the genotype is determined using a hybridization-based method, an enzyme-based method, a post-amplification method based on physical properties of DNA, a sequencing method, or a combination of two or more thereof.

14. The method of claim 8, wherein the genotype is determined to be a minor allele, major allele, or heterozygous allele using at least one nucleic acid analysis technique selected from: DNA sequencing, restriction enzyme digest, polymerase chain reaction (PCR), hybridization, real-time PCR, reverse transcriptase PCR, ligase chain reaction, and a combination of two or more thereof.

15. The method of claim 8 further comprising:
providing instructions to provide a composition comprising arginine based on the genotype of the argininosuccinate synthase gene of the subject.
